(19) United States
(12) Patent Application Publication
(10) Pub. No.: US 2026/0155253 A1
(43) Pub. Date: Jun. 4, 2026

(54) PREDICTION OF STAGE AND SURVIVAL USING ISOFORM EXPRESSION IN GASTRIC ADENOCARCINOMA

(71) Applicant: UNIVERSITY OF SOUTH FLORIDA, Tampa, FL (US)

(72) Inventors: Paul KUO, Tampa, FL (US); Yifan LIANG, Tampa, FL (US); Kyle LIEN, Tampa, FL (US)

(21) Appl. No.: 19/408,197

(22) Filed: Dec. 3, 2025

Related U.S. Application Data
(60) Provisional application No. 63/727,513, filed on Dec. 3, 2024.

(51) Int. Cl. G16H 50/20; G16H 10/40; G16H 10/60

(52) U.S. Cl. CPC G16H 50/20; G16H 10/40; G16H 10/60

(57) ABSTRACT

Various processes, methods and systems are provided herein for assisting patients, medical providers and other personnel in predicting cancer stage and cancer survival. The systems and methods include determining an indication of a preliminary cancer diagnosis of a given type of cancer for a patient, receiving results of transcriptional sequencing analysis, extracting a set of isoform expression information from transcriptomic data, processing a cancer stemness isoform dataset for a patient, and outputting an indication of the cancer stage prediction or the survival prediction.

### CROSS-REFERENCE TO RELATED APPLICATIONS

This application claims priority to U.S. provisional patent application Nos. 63/727,513 filed Dec. 3, 2024, the entire contents of which are incorporated herein by reference.

### STATEMENT REGARDING FEDERALLY SPONSORED RESEARCH

N/A

### BACKGROUND

Gastric adenocarcinoma is the most common type of stomach cancer. The survival rates of gastric adenocarcinoma are highly dependent on cancer stage classification. Despite significant strides in targeted therapies and surgical interventions to improve a patient's prognosis, the need for precise and personalized predictions regarding cancer stage and cancer survival is paramount and can profoundly influence clinical decision-making for patients diagnosed with gastric adenocarcinoma.

Cancer stemness describes the capacity of cancer stem cells (CSCs) for differentiation into various cell types and self-renewal. While CSCs form only a fraction of the total cell population within tumors, they have a meaningful role in tumor growth and dissemination. Alternative splicing, whereby a single gene can produce multiple mRNA variants and a variety of isoforms, maintains cancer stemness properties in CSCs. Despite databases of isoform expression and gene signatures of cancer cells being available, it remains unknown how expression of specific isoforms impacts cancer stage and cancer survival for patients with gastric adenocarcinoma. What are needed are systems and methods that provide real-time, accurate assessments of how specific isoform expressions relate to cancer stage and survival predictions, enabling appropriate diagnoses and guiding clinical treatments for patients with gastric adenocarcinoma.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any of all aspects of the disclosure. Its sole purpose is to present some concepts of one or more aspects of the disclosure in a simplified form as a prelude to the more detailed description that is presented later.

In one aspect, the present disclosure provides a system for guiding clinical decision-making for potential cancer patients comprising: a first memory having stored thereon an electronic medical record database framework including medical data for a given patient; a communication system connected to the memory to allow credentialed users at remote workstations to input new medical data into and retrieve existing medical data from the first memory; a processor communicatively connected to the first memory and a second memory; and wherein the second memory has software instructions stored thereon which, when executed by the processor, cause the system to: determine an indication of a preliminary cancer diagnosis of a given type of cancer for the given patient from the medical data stored on the first memory; confirm an order for a biopsy and associated transcriptional sequencing analysis of the biopsy; receive results of the transcriptional sequencing analysis, the results comprising transcriptomic data representative of isoform and gene expression of cells of the biopsy; extract a set of isoform expression information from the transcriptomic data, based on one or more gene cancer stemness-related signatures specific to the preliminary cancer diagnosis, to create a cancer stemness isoform dataset for the patient; process the cancer stemness isoform dataset for the patient using a first machine learning model to obtain a baseline current cancer stage prediction for the patient, the first machine learning model having been trained on cancer patient data from patients confirmed to have the given type of cancer and configured to output a prediction of cancer stage classification; process the cancer stemness isoform dataset for the patient using a second machine learning model to obtain baseline survival prediction information for the patient, the first machine learning model having been trained on cancer patient data from patients confirmed to have the given type of cancer and configured to output survival prediction information over a period of time after diagnosis; store the current cancer stage prediction and the survival prediction information in the first memory with the medical data for the given patient; output via the communication system an indication to a healthcare provider of the cancer stage prediction, the survival prediction information and at least one a treatment recommendation based thereon.

In another aspect the present disclosure provides a method for guiding clinical decision-making for potential cancer patients, the method comprising: receiving medical data for a given patient, determining an indication of a preliminary cancer diagnosis of a given type of cancer for the given patient from the medical data; confirming an order for a biopsy and associated transcriptional sequencing analysis of the biopsy; receiving results of the transcriptional sequencing analysis, the results comprising transcriptomic data representative of isoform and gene expression of cells of the biopsy; extracting a set of isoform expression information from the transcriptomic data, based on one or more gene cancer stemness-related signatures specific to the preliminary cancer diagnosis, to create a cancer stemness isoform dataset for the patient; processing the cancer stemness isoform dataset for the patient using a first machine learning model to obtain a baseline current cancer stage prediction for the patient, the first machine learning model having been trained on cancer patient data from patients confirmed to have the given type of cancer and configured to output a prediction of cancer stage classification; processing the cancer stemness isoform dataset for the patient using a second machine learning model to obtain baseline survival prediction information for the patient, the first machine learning model having been trained on cancer patient data from patients confirmed to have the given type of cancer and configured to output survival prediction information over a period of time after diagnosis; and output an indication to a healthcare provider of the cancer stage prediction, the survival prediction information and at least one a treatment recommendation based thereon.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments, it should be understood that such example embodiments can be implemented in various devices, systems, and methods.

### BRIEF DESCRIPTION OF THE DRAWINGS

FIG. 1 is a flowchart illustrating an example of obtaining a result of a cancer stage classification or a result of cancer survival prediction based on the outcome of a cancer stage classification or cancer survival prediction model.

FIG. 2 is a flowchart illustrating an example of a process for generating a machine learning model.

FIG. 3 is a flowchart illustrating an example generating an alert that a patient's cancer stage classification or a patient's cancer survival prediction has changed based on the outcome of a model.

FIG. 4 is a block diagram conceptually illustrating a system for determining a cancer stage classification or a cancer survival prediction.

FIG. 5 presents a distribution of unique isoforms for each patient, according to some embodiments.

FIG. 6 presents performance comparisons between cancer stage classification and cancer survival models, according to some embodiments.

FIG. 7 presents a functional analysis of genes associated with features of the cancer survival model, according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, certain known structures and components are shown in block diagram form to avoid obscuring such concepts.

Various embodiments, features, and examples of the present disclosure can also be found in the attached appendices, comprising academic articles which describe the inventors' work. These articles support the breadth of and are not limiting of the scope of the present disclosure.

### Example Process—Cancer Stage & Cancer Survival

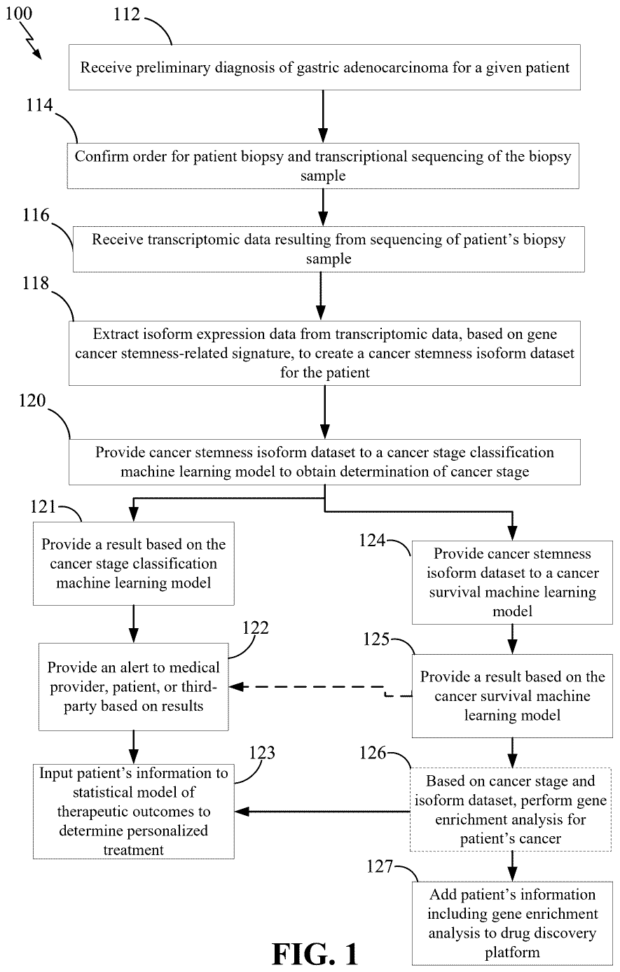

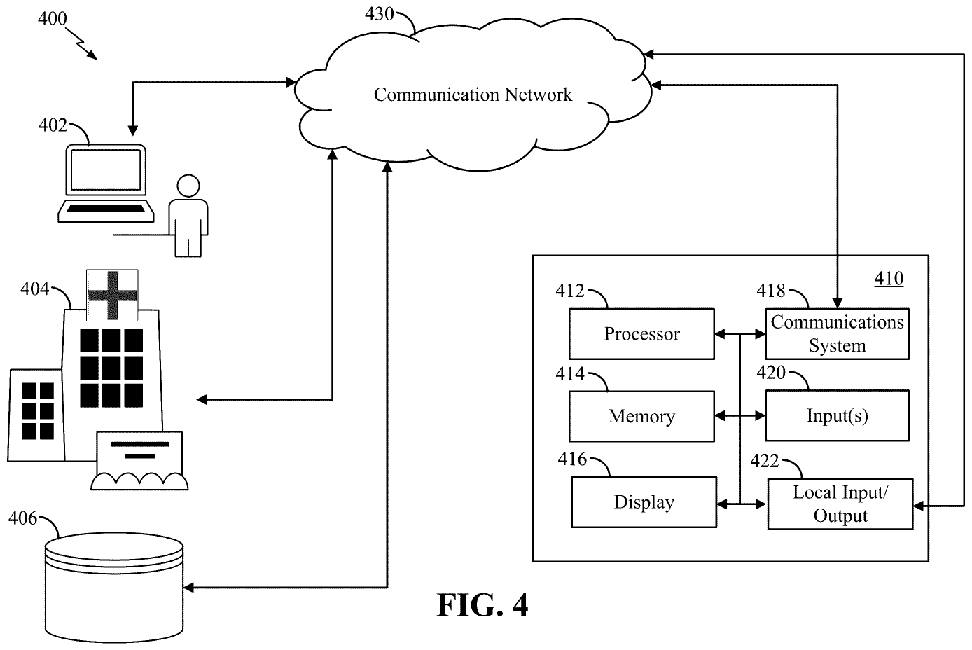

FIG. 1 is a flow diagram illustrating an example of process 100 for improved determination of a potential cancer patient's cancer stage, the potential patient's survival prediction, or both, based on objective, and potentially single-source data. Thus, a result of a cancer stage classification, or a result of cancer survival prediction, or both, can further be utilized for a variety of tasks generally falling within the category of clinical decision-making (as further described below) and can be updated based on changes in the patient's treatment and symptoms. As described below, a particular implementation can omit some or all illustrated features/steps, may be implemented in some embodiments in a different order, and may not require some illustrated features to implement all embodiments. In some examples, an apparatus (e.g., processor 412 with memory 414) in connection with FIG. 4 can be used to perform example process 100. However, it should be appreciated that any suitable apparatus or means for carrying out the operations or features described below may perform process 100.

In some embodiments, process 100 can be used to help determine a patient's stage of cancer. In some embodiments, process 100 can be used to help determine a patient's stage of gastric adenocarcinoma. For example, process 100 can be used to determine if a patient has gastric adenocarcinoma stage I, stage II, stage, III, or stage, IV, or is between any stages. In some embodiments, process 100 can also be used to help determine a patient's time of survival following a cancer diagnosis. For example, process 100 can provide a result for an approximate time-to-death timeframe for a patient.

At step 112, process 100 can receive a preliminary diagnosis of gastric adenocarcinoma for a given patient. In some examples, a medical provider or assistant may input the preliminary diagnosis of gastric adenocarcinoma into a graphical user interface, or the preliminary diagnosis may be obtained from an electronic medical record, a medical provider database, or extracted from a scan or patient chart, or may be obtained from another user or source. In some embodiments, a preliminary diagnosis of gastric adenocarcinoma can be obtained from histological analysis of a biopsy sample.

As one specific example, a medical records or healthcare software platform may provide an interface for a provider or healthcare team member into which a preliminary diagnosis can be entered. This may take the form of a suspected diagnosis code, a narrative explanation for why the healthcare team believes the patient is suspected of having a potential cancer, or a guided workflow based on known or standard of care diagnostic standards, risk factors, and/or symptom confirmation. In some embodiments, an agentic AI tool may be utilized to parse provider notes and match healthcare team observations, test results, and patient data to a set of diagnostic indicators. In further embodiments, the information to be input may be required by an insurer, payer, or risk management protocol in order to qualify the patient for further analysis and/or treatment.

At step 114, process 100 can confirm an order or prescription for a biopsy has been entered for the patient. In some embodiments, this may be a step that is permitted only after step 112 has confirmed a sufficient likelihood of cancer, or only if a provider has ordered a given cancer treatment (but a diagnostic confirmation is first desired or required). In further embodiments, step 114 may also entail confirming that the order for biopsy will generate a suitable sample for transcriptional analysis, that a transcriptional analysis or sequencing has been also ordered, and/or that the type of transcriptional analysis ordered is capable of providing suitable transcriptomic data of the biopsy for further steps. In some examples, a medical provider or assistant may input an order of a biopsy and transcriptional sequencing of the biopsy sample. In some embodiments, histological staining of a biopsy sample may be performed and the results and interpretation of the histology stain are inserted into a patient's medical record. In other examples, the order of a biopsy and transcriptional sequencing may be obtained from an electronic medical record, a medical provider database, or extracted from a scan or patient chart, or may be obtained from another user or source.

At step 116, process 100 can receive transcriptomic data resulting from sequencing of the patient's biopsy sample. Thus, step 116 may further include confirming that appropriate steps were taken to enrich or extract the mRNA from the biopsy sample and properly prepare it for sequencing (e.g., cDNA conversion, etc.). In some cases, more than one sample from a biopsy, or samples from biopsies taken from multiple tumors or lesions may all be processed in a like manner so that the transcriptomic data provide multiple examples. In some implementations of process 100, the transcriptomic data comprises the sequencing data of the mRNA transcripts from the biopsy sample(s). In some examples, the transcriptomic data comprises isoform and gene expression data. In some examples, the isoform expression and gene expression data is quantified as RSEM (RNAseq by Expectation-Maximization) normalized data. In some embodiments, isoforms are coded starting with uc0, which represents UCSC gene identifications. Step 116 may, thus, also comprise detection of isoform and gene expression data and/or normalization of the transcriptomic data.

At step 118, process 100 can process the transcriptomic data to extract a cancer-specific set of isoform expression information, based on one or more gene cancer stemness-related signatures, to create a cancer stemness isoform dataset for the patient. In some examples, the isoform expression data is determined and extracted by comparing the sequencing data of the patient's biopsy sample from a curated gene signature database consisting of a 109 gene cancer stemness signature along with 10 “canonical” cancer stemness markers. For instance, the isoform expression data in a patient's biopsy sample can be compared to the isoform expression data in a database to determine the types and quantities of isoforms found in the patient's sample. In other examples, the isoform expression data is determined and extracted by comparing the sequencing data of the patient's biopsy sample to the genes from an online gene signature database, such as The Cancer Genome Atlas Project (TCGA).

At step 120, process 100 applies the cancer stemness isoform dataset to a cancer stage classification machine learning model to obtain a determination of cancer stage. In some examples, the cancer stemness isoform datasets may be processed using a specially trained classification model based on support vector machine (SVM), ElasticNet, GradientBoosting, and/or Random Forest machine learning algorithms.

At step 121, process 100 may provide a result based on the cancer stage classification machine learning model. For example, step 121 of process 100 may output a result that a patient has stage I, stage II, stage III, or stage IV cancer. In some embodiments, step 121 of process 100 may output a result classifying the cancer as low stage (Stage I) or high stage (II, III, and IV). In some embodiments, the result will include a confidence score for each prediction. In other examples, step 121 of process 100 may provide a result that a patient has a cancer indicative of a level between stages (e.g. stage II-stage III) or a confidence level for each of the four stages (or for only the top 2 most likely stages), along with a confidence score for the prediction. Alternatively, the output categories may further include a sixth and/or seventh type, indicating that the patient does not have the type of cancer that was preliminarily diagnosed and/or indicating that the patient does have the type of cancer that was preliminarily diagnosed. In this fashion, if confidence levels among the possible 5 cancer stage outputs are close in value, users can determine if the patient even has that cancer or not, or if the model at least agrees that the patient has that type of cancer but is having difficulty distinguishing among stages. In some embodiments, this result can be utilized by a system operating process 100 to guide the medical provider's choice of care, such as to perform a specific type of surgery. In other embodiments, this result can be utilized by a system operating process 100 to guide a medical provider to administer a particular kind of chemotherapy for treatment of the cancer. In some embodiments, this result can provide information to an insurance company regarding insurance coverage decisions for the patient.

At step 122, process 100 may provide a notification to a medical provider, a patient, or a third party based on the results provided from step 121. For example, step 122 of process 100 could provide an alert to a medical provider, a patient, or a third-party of the cancer stage results. In some examples, this alert can be transmitted to an electronic medical record, a medical provider, a patient, or a third-party such as an insurance company.

At step 123, process 100 can input the patient's information to a statistical model of therapeutic outcomes to determine a personalized treatment plan. For example, if a patient has a particular set of isoforms being expressed (or expressed at certain levels) and receives a classification of stage I cancer, the patient will be offered a selection of specific therapeutics and treatment options for stage I cancer from a medical provider. However, if a patient has a particular set of isoforms and receives a classification of stage IV cancer, the patient will be offered a selection of therapeutics and treatment options for stage IV cancer.

In other words, because the isoform expression data for a given patient is objective and consistent in scope, some implementations can leverage this data together with patient treatment and progression information, to develop recommendations for particular forms of treatment that may work best for a given gene expression of a given cancer. This can allow for more personalized treatment as well as for updating and tailoring statistical predictors that match a patient's specific isoform expression profile with similar profiles of past patients and identify which treatments tended to work best for that type of patient.

At step 124, process 100 applies the cancer stemness isoform dataset to a cancer survival machine learning model to obtain a determination of a patient's time of survival following a cancer diagnosis. For example, process 100 can provide a result for an approximate time-to-death timeframe for a patient. In some examples, the cancer stemness isoform datasets may be processed using Survival SVM with linear and radial basic function (RBF) kernels, Random Survival Forest, GradientBoosting Survival, and ElasticNet Survival machine learning algorithms.

At step 125, process 100 can provide a result based on the cancer survival machine learning model. For example, step 125 of process 100 may provide a result that a patient has a life expectancy of a certain timeframe. For instance, step 125 of process 100 may provide a result that a patient has a life expectancy of six-months.

At step 122, process 100 may provide an alert to a medical provider, a patient, or a third party based on the results provided from step 125. For example, step 122 of process 100 could provide an alert to a medical provider, a patient, or a third-party of the patient's time of survival following a cancer diagnosis (cancer survival). In some examples, this alert can be transmitted to an electronic medical record, a medical provider, a patient, or a third-party such as an insurance company.

At step 126, process 100 may optionally perform gene enrichment analysis for a patient's cancer based on the cancer stage and isoform dataset. In some embodiments, Feature importance can be extracted using the GradientBoosting Survival model, and gene enrichment analysis performed on the genes corresponding to a sample of 100 features (such as those found in Table 1) to determine functional importance using Gene Ontology (GO) term enrichment and Kyoto Encyclopedia of Genes and Genomes (KEGG) pathway analyses. In some embodiments, gene enrichment analysis can be performed on other sample features based on an automated or curated database. In some embodiments, gene enrichment analysis will indicate types of gene sets that are significantly enriched based on the patient's isoforms. For instance, a patient may have isoforms that indicate gene sets involving cell cycle, cell metabolism, and nucleotide activity are significantly enriched. At step 126, process 100 may also proceed to step 123 and input the patient's information to a statistical model of therapeutic outcomes to determine a personalized treatment plan. For example, in one embodiment, if a patient has a particular set of isoforms that indicates genes involving disruptions of the cell cycle are a potential source of the cancer, the patient may be given targeted therapeutics to address the cell cycle disruptions. In another example, if a patient has a particular set of isoforms that show enhanced cell metabolism of cancer cells, the patient may be administered a personalized and specific treatment to disrupt the cancer cell metabolic cycle.

At step 127, process 100 may provide the patient's isoform dataset, result of life expectancy and result of gene enrichment analysis to a drug discovery platform. For example, if a patient has a particular set of isoforms that show enhanced cell metabolism of cancer cells based upon the gene enrichment analysis, the information can be conveyed to a database that stores information regarding drug discovery for particular genes related to cancer cell metabolism. In some examples, the results generated by the survival machine learning model and transmitted to a drug discovery platform at step 127 can identify which genes and specific isoforms most frequently contribute to gastric adenocarcinoma, supporting future drug development that targets proteins derived from these enriched genes.

### Example Machine Learning Model Training Process

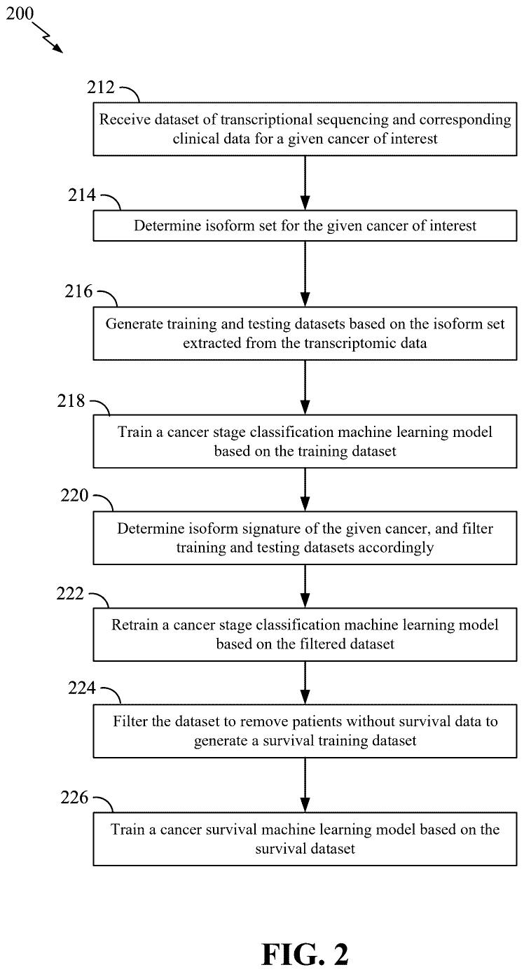

FIG. 2 is a flow diagram illustrating a process for training a machine learning model. As described below, a particular implementation can omit some or all illustrated features/steps, may be implemented in some embodiments in a different order, and may not require some illustrated features to implement all embodiments. In some examples, an apparatus (e.g., processor 412 with memory 414) in connection with FIG. 4 can be used to perform example process 200. However, it should be appreciated that any suitable apparatus or means for carrying out the operations or features described below may perform process 200.

At step 212, process 200 receives a dataset of transcriptional sequencing and corresponding clinical data for a given cancer of interest. In some embodiments, the transcriptomic data comprises the sequencing data of the mRNA transcripts from the biopsy sample(s). In some examples, the transcriptomic data comprises isoform and gene expression data from the biopsy sample. In some embodiments, the clinical data may include CT findings, PET scans, endoscopic ultrasounds scans (EUS), blood markers (i.e. carcinoembryonic antigen [CEA]) age, sex, history of smoking, weight, diet, administration of chemotherapy, surgery, or any indications associated with a clinical record of a cancer patient.

At step 214, process 200 determines the isoform set for the given cancer of the patient. In some examples, the isoform expression data is determined and extracted by comparing the isoform expression data of the patient's biopsy sample to a cancer-type specific gene signature. For example, for gastric adenocarcinoma, the applicable gene signature could be a curated gene signature comprising or consisting of a 109-gene cancer stemness signature along with 10 “canonical” cancer stemness markers. For instance, the isoform expression data in a patient's biopsy sample can be compared to the isoform expression data in one or more signatures for different likely cancer types. In other examples, the isoform expression data is determined by comparing the isoform expression data of the patient's biopsy sample to the genes from an online gene signature database, such as The Cancer Genome Atlas Project (TCGA) database.

In some embodiments, the cancer-specific signature may be developed in advance by collecting a preliminary list of genes known to be associated with a specific cancer type or a general category of cancer, and adding markers that are well known to be associated with cancer. The markers can comprise widely recognized markers of cancer stem cells (CSCs), such as OCT4, SOX2, NANOG, CD44, CD133, etc., to serve as reference points or validation markers to confirm that the signature aligns with known biology. Then, this list can be compared to a sample population known to have a given type of cancer and curated or tailored through a variety of means. For example, various single or multi-variate regressions and/or statistical methods (e.g., LASSO, recursive feature elimination, regressions, gene interaction networks, etc.) may be used to identify the most relevant genes, relationships among genes, or similar features of the signature as relates to a given cancer.

At step 216, process 200 generates training and testing datasets based on the isoform set extracted from the transcriptional sequencing data. In some examples, the training and testing datasets are randomly divided at a 6:4 split.

At step 218, process 200 trains a cancer stage classification machine learning model based on the training dataset. The cancer stage classification learning model may be utilized in step 120 of process 100. In some examples, the cancer stage classification model can utilize Support vector machine (SVM), ElasticNet, GradientBoosting, and Random Forest algorithms either individually, or in specific combinations of each algorithm.

At step 220, process 200 determines the isoform signature of the given cancer and filtering the training and testing datasets accordingly. For example, if particular isoforms related to cell cycle regulation are not provided in the isoform signature of the given cancer, these isoforms are be removed from the training and testing datasets.

At step 222, process 200 retrains a cancer stage classification machine learning model based on the filtered datasets. In some examples, the cancer stage classification model is retrained using Support vector machine (SVM), ElasticNet, GradientBoosting, and Random Forest algorithms either individually, or in specific combinations of each algorithm.

At step 224, process 200 filters the dataset to remove patients without survival data to generate a survival training dataset. For example, patients without survival data are removed from the analysis to generate an accurate survival machine learning model.

At step 226, process 200 trains a cancer survival machine learning model based on the training dataset. In some examples, the cancer survival machine learning model can utilize Survival SVM with linear and radial basic function (RBF) kernels, Random Survival Forest, GradientBoosting Survival, and ElasticNet Survival algorithms, either individually or in specific combinations of each algorithm.

### Example Process—Using the Model to Update Cancer Classification and Survival Predictions

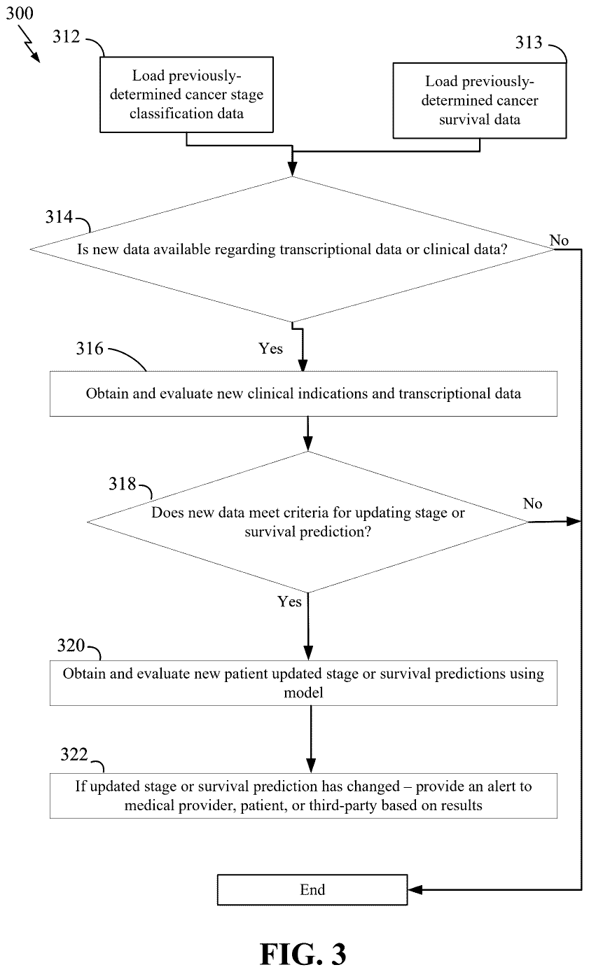

Referring now to FIG. 3, a flow diagram is shown for example process 300 to re-evaluate a patient's cancer stage classification or a patient's time of survival following a cancer diagnosis (cancer survival) in response to surgery, therapy, chemotherapy, diet change, exercise, or any type of treatment or regimen directed to improving the patient's prognosis. As described below, a particular implementation can omit some or all illustrated features/steps, may be implemented in some embodiments in a different order, and may not require some illustrated features to implement all embodiments. In some examples, an apparatus (e.g., processor 412 with memory 414) in connection with FIG. 4 can be used to perform example process 400. However, it should be appreciated that any suitable apparatus or means for carrying out the operations or features described below may perform process 300.

At step 312 and 313, the process 300 loads the previously-determined cancer stage classification data or the previously-determined cancer survival data. For example, an initial set of data may have been previously obtained such as in relation to a process of FIG. 1. In some embodiments, this may involve accessing previously-stored data in a patient's electronic medical record, a patient device 402, a facility, a clinic, a hospital 404, any other suitable data source 406.

At step 314, the process 300 determines if new data is available for any clinical data or transcriptional indications. In some embodiments, clinical data may include CT findings, PET scans, endoscopic ultrasounds scans (EUS), blood markers (i.e. carcinoembryonic antigen [CEA]) age, sex, history of smoking, weight, diet, administration of chemotherapy, surgery, or any indications associated with a clinical record of a cancer patient. In some embodiments, process 300 may periodically monitor the electronic medical record of a given patient to determine whether any new clinical indications or transcriptomic data has been entered since the last time the cancer stage classification or cancer survival prediction was determined.

At step 316, the process 300 obtains and evaluates the new data (e.g. new values of clinical data or transcriptomic data). For example, the process may obtain and extract the data from an updated CT scan.

At step 318, the process 300 evaluates if the new data meets the criteria for updating the cancer stage prediction or cancer survival prediction. Such criteria may include factors such as: duration of time since the last update to the patient clinical data or transcriptomic data, obtaining of transcriptomic data of a new biopsy sample, new clinical data such as an updated CT scan, PET Scan, EUS, blood marker indications, indication of changes in diet, administration of a specific pharmacotherapy, administration of chemotherapy, administration of chemo-radiation, or if surgery was performed to remove the cancer (surgical resection).

In some embodiments, the process 300 determines the new data meets the criteria for updating the cancer stage classification or cancer survival prediction. In these cases, at step 320 of process 300, the process will re-determine the cancer stage classification prediction or the cancer survival prediction using the cancer stage classification machine learning model or the cancer survival machine learning model, based on the new data.

At step 322, the process 300 will provide an alert to a medical provider, patient, or a third-party based on the results if the cancer stage classification prediction or the cancer survival prediction has changed. For example, an alert could be provided within a patient's electronic medical record and a message communicated to a medical provider, a patient, or a third-party (such as an insurance company) of the updated cancer stage classification prediction or the cancer survival prediction.

### Example Hardware Integration of Model

FIG. 4 shows a block diagram illustrating a system for determining a cancer stage classification or a cancer survival prediction, according to some embodiments. As shown in FIG. 4, computing device 410 can receive a preliminary diagnosis of gastric adenocarcinoma for a given patient. Computing device 410 can additionally receive transcriptomic data resulting from sequencing of the patient's biopsy sample. Computing device 410 can extract isoform expression data from the transcriptomic data based on a gene cancer stemness-related signature, and create a cancer stemness isoform dataset for the patient. Computing device 410 can additionally provide a cancer stemness isoform dataset to a cancer stage classification machine learning model to obtain determination of cancer stage, and provide a result based on the cancer stage classification machine learning model. For example, computing device 410 may provide a result that a patient has stage I, stage II, stage III, or stage IV cancer. In some embodiments, computing process 410 may output a result classifying the cancer as low stage (Stage I) or high stage (II, III, and IV). In some embodiments, the result will include a confidence score for each prediction. In other examples, computing process 410 may output a result that a patient has a cancer indicative of a level between stages (e.g. stage II-stage III) or a confidence level for each of the four stages (or for only the top 2 most likely stages), along with a confidence score for the prediction. Alternatively computing process 410 may output a result indicating that the patient does not have the type of cancer that was preliminarily diagnosed and/or indicating that the patient does have the type of cancer that was preliminarily diagnosed. In some embodiments, computing device 410 may provide an alert to medical provider, patient, or third-party based on the results, and input a patient's information to a statistical model of therapeutic outcomes to determine a personalized treatment plan.

In other embodiments, computing device 410 may provide a cancer stemness isoform dataset to a cancer survival machine learning model and provide a result based on the cancer survival machine learning model. For example, computing device 410 may provide a result that a patient has a life expectancy of a certain timeframe. For instance, computing device 410 may provide a result that a patient has a life expectancy of six-months based on the model. Computing device 410 may optionally perform gene enrichment analysis for a patient's cancer based on the cancer stage and dataset. In some embodiments, computing device 410 may also input the patient's information to a statistical model of therapeutic outcomes to determine a personalized treatment plan. In some embodiments, computing device 410 may also add a patient's information including gene enrichment analysis to a drug discovery platform.

In some examples, computing device 410 can provide an updated cancer stage or cancer survival prediction as described in process 300 and in connection with FIG. 3.

In some examples, computing device 410 can include processor 412. In some embodiments, the processor 412 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc. Processor 412 may be located within a local (to the user) device (such as a mobile device), may be associated with a system hosting a patient medical record application, may be associated with a system providing information to physicians, may be part of a cloud-based resource, or otherwise, depending on the particular embodiment.

In further examples, computing device 410 can further include a memory 414. The memory 414 can include any suitable storage device or devices that can be used to store suitable data and instructions that can be used, for example, by the processor 412 to receive a first plurality of entries corresponding to a plurality donor factor and a second plurality of entries corresponding to a plurality of recipient factors. The memory 414 can include any suitable volatile memory, non-volatile memory, storage, or any suitable combination thereof. For example, memory 414 can include random access memory (RAM), read-only memory (ROM), electronically-erasable programmable read-only memory (EEPROM), one or more flash drives, one or more hard disks, one or more solid state drives, one or more optical drives, cloud-based resources, etc. In some embodiments, the processor 412 can execute at least a portion of processes 100 and/or 200, and/or 300 as described above in connection with FIG. 1 or 2, or 3.

In further examples, computing device 410 can further include communications system 418. Communications system 418 can include any suitable hardware, firmware, and/or software for communicating information over communication network 430 and/or any other suitable communication networks. For example, communications system 418 can include one or more transceivers, one or more communication chips and/or chip sets, etc. In a more particular example, communications system 418 can include hardware, firmware and/or software that can be used to establish a Wi-Fi connection, a Bluetooth connection, a cellular connection, an Ethernet connection, a local network, etc.

In further examples, computing device 410 can receive or transmit information (e.g., from or to a patient device 402, a facility, a clinic, a hospital 404, any other suitable data source 406, and/or any other suitable system) over a communication network 430. In some examples, the communication network 430 can be any suitable communication network or combination of communication networks. For example, the communication network 430 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 430 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links, Bluetooth links, cellular links, etc.

In some examples, the computing device 410 can further transmit an output connection 422 to a patient device 402, a facility, a clinic, a hospital 404, any other suitable data source 406, The output 422 connection may be part of or rely upon a network connection such as the communication network 430, but alternatively may be a separate connection such as, e.g., a private connection to a healthcare organization's electronic medical record system or may include other connections such as an email server. The form of output connection 422 may depend upon the form of data to be provided to a user as well as where the computing device 410 resides. As another example, if the computing device 410 is hosted by a healthcare organization or clinic, the output may comprise all or a portion of a user interface directed to the treating provider. In some embodiments, the output connection 422 can transmit a cancer stage classification result, a result that a patient has a life expectancy of a certain timeframe (cancer survival), or an alert to a medical provider, patient or third-party based on the cancer stage classification or cancer results.

In further examples, the cancer stage classification result, life expectancy, or the alert to a medical provider, patient or third-party, or any output from computing device 410 can be transmitted to another system or device over the communication network 430.

In further examples, computing device 410 can further include a display 416 and/or one or more inputs 420. In some embodiments, the display 416 can include any suitable display devices, such as a computer monitor, a touchscreen, a television, an infotainment screen, etc. to display a report about the cancer stage classification result, life expectancy, results of gene enrichment analysis, or any suitable information relating to the patient's cancer prognosis. In further embodiments, the input(s) 420 can include any suitable input devices and/or sensors that can be used to receive user input, such as a keyboard, a mouse, a touchscreen, a microphone, etc.

### EXAMPLE IMPLEMENTATIONS

The foregoing systems and methods may be implemented in a variety of forms for a variety of further applications.

For example, in one implementation, the foregoing systems and methods may be applied to providing a result of a cancer stage classification for a patient. In another implementation, the foregoing systems and methods may transmit an alert to a medical provider, patient, or third-party based on the results. The cancer stage classification results provided by the foregoing systems and methods may allow medical providers to determine a personalized treatment plan based on the results. For instance, the cancer stage classification results provided by the foregoing systems and methods may be provided to a statistical model of therapeutic outcomes to determine a personalized treatment plan for the patient. In some examples, the cancer stage classification results provided by the foregoing systems and methods may suggest indications regarding the success of a particular treatment. For example, if the results indicate a patient has stage II cancer, this data suggests that treating the cancer using therapies typically directed to stage I cancer may be unsuccessful.

For example, in another implementation, the foregoing systems and methods may provide a result indicating a patient's time of survival following a cancer diagnosis. For instance, this data may be useful to insurance companies and medical providers when considering coverage options of a treatment plan. This data may also be useful to family members regarding care for the patient, such as by providing a timeframe to contact hospice care.

In another implementation, the foregoing systems and methods may provide a result indicating the specific enriched genes of a patient's cancer. For instance, the results provided by the foregoing systems and methods could be used in drug-discovery platforms based upon the enriched genes. For example, drugs could be developed and applied to target or interrupt cancer-causing or cancer-associated proteins or pathways based upon the findings of the enriched genes. Furthermore, the foregoing systems and methods can help determine the efficacy of treatments used against a particular stage of cancer. For instance, after a patient with a particular set of isoforms has followed a prescribed treatment plan, the systems and methods of the present disclosure can evaluate a second set of clinical or transcriptional data as indicated in FIG. 3. If the cancer stage or cancer survival prediction has changed in response to a specific personalized treatment plan, this indicates the efficacy of the current treatments based on the isoforms from a particular patient. The systems and methods of the present disclosure provide data for future regression analysis to determine appropriate treatments and pharmacotherapies based on the expression of specific isoforms.

In another implementation, the present disclosure provides a system for guiding clinical decision-making for potential cancer patients. In some embodiments, the system comprises a first memory having stored thereon an electronic medical record database framework including medical data for a given patient; a communication system connected to the memory to allow credentialed users at remote workstations to input new medical data into and retrieve existing medical data from the first memory; a processor communicatively connected to the first memory and a second memory; and wherein the second memory has software instructions stored thereon. In some embodiments, the software instructions, when executed by the processor, cause the system to: determine an indication of a preliminary cancer diagnosis of a given type of cancer for the given patient from the medical data stored on the first memory; confirm an order for a biopsy and associated transcriptional sequencing analysis of the biopsy; receive results of the transcriptional sequencing analysis, the results comprising transcriptomic data representative of isoform and gene expression of cells of the biopsy; extract a set of isoform expression information from the transcriptomic data, based on one or more gene cancer stemness-related signatures specific to the preliminary cancer diagnosis, to create a cancer stemness isoform dataset for the patient; process the cancer stemness isoform dataset for the patient using a first machine learning model to obtain a baseline current cancer stage prediction for the patient, the first machine learning model having been trained on cancer patient data from patients confirmed to have the given type of cancer and configured to output a prediction of cancer stage classification; process the cancer stemness isoform dataset for the patient using a second machine learning model to obtain baseline survival prediction information for the patient, the first machine learning model having been trained on cancer patient data from patients confirmed to have the given type of cancer and configured to output survival prediction information over a period of time after diagnosis; store the current cancer stage prediction and the survival prediction information in the first memory with the medical data for the given patient; output via the communication system an indication to a healthcare provider of the cancer stage prediction, the survival prediction information and at least one a treatment recommendation based thereon.

In another implementation, the present disclosure provides a provides a method for guiding clinical decision-making for potential cancer patients. In some embodiments, the method comprises receiving medical data for a given patient, determining an indication of a preliminary cancer diagnosis of a given type of cancer for the given patient from the medical data; confirming an order for a biopsy and associated transcriptional sequencing analysis of the biopsy; receiving results of the transcriptional sequencing analysis, the results comprising transcriptomic data representative of isoform and gene expression of cells of the biopsy; extracting a set of isoform expression information from the transcriptomic data, based on one or more gene cancer stemness-related signatures specific to the preliminary cancer diagnosis, to create a cancer stemness isoform dataset for the patient; processing the cancer stemness isoform dataset for the patient using a first machine learning model to obtain a baseline current cancer stage prediction for the patient, the first machine learning model having been trained on cancer patient data from patients confirmed to have the given type of cancer and configured to output a prediction of cancer stage classification; processing the cancer stemness isoform dataset for the patient using a second machine learning model to obtain baseline survival prediction information for the patient, the first machine learning model having been trained on cancer patient data from patients confirmed to have the given type of cancer and configured to output survival prediction information over a period of time after diagnosis; and output an indication to a healthcare provider of the cancer stage prediction, the survival prediction information and at least one a treatment recommendation based thereon.

### ANALYSES, EXPERIMENTS, AND VALIDATION STUDIES

Materials and Methods: Transcriptional and clinical data for 392 Gastric adenocarcinoma (STAD) patients were downloaded from the TCGA Splicing Variants database site (TSVd), a web-tool to explore mRNA alternative-splicing and clinical data based on TCGA samples. Data downloaded includes isoform expression, clinical sample types, and survival data. Isoform expression and gene expression are quantified as RSEM (RNAseq by Expectation-Maximization) normalized data. Isoforms are coded starting with uc0, which represents UCSC gene identifications.

### Predictive Models

Expression of 380 isoforms across a 119 gene cancer stemness-related signature were collected and was used as features for ML analysis. This includes a 109 gene cancer stemness-related signature, as well as 10 additional well-established cancer stem cell markers (Table 1).

TABLE 1
119 gene cancer stemness-related signature.

| | |
|---|---|
| DNMT3B | RPF2 |
| PFAS | APLP1 |
| XRCC5 | DACT1 |
| HAUS6 | PDHB |
| TET1 | C14orf119 |
| IGF2BP1 | DTD1 |
| PLAA | SAMM50 |
| TEX10 | CCL26 |
| MSH6 | MED20 |
| DLGAP5 | UTP6 |
| SKIV2L2 | RARS2 |
| SOHLH2 | ARMCX2 |
| RRAS2 | RARS |
| PAICS | MTHFD2 |
| CPSF3 | DHX15 |
| LIN28B | HTR7 |
| IPO5 | MTHFD1L |
| BMPR1A | ARMC9 |
| ZNF788 | XPOT |
| ASCC3 | IARS |
| FANCB | HDX |
| HMGA2 | ACTRT3 |
| TRIM24 | ERCC2 |
| ORC1 | TBC1D16 |
| HDAC2 | GARS |
| HESX1 | KIF7 |
| INHBE | UBE2K |
| MIS18A | SLC25A3 |
| DCUN1D5 | ICMT |
| MRPL3 | UGGT2 |
| CENPH | ATP11C |
| MYCN | SLC24A1 |
| HAUS1 | EIF2AK4 |
| GDF3 | GPX8 |
| TBCE | ALX1 |
| RIOK2 | OSTC |
| BCKDHB | TRPC4 |
| RAD1 | HAS2 |
| NREP | FZD2 |
| ADH5 | TRNT1 |
| PLRG1 | MMADHC |
| ROR1 | SNX8 |
| RAB3B | CDH6 |
| DIAPH3 | HAT1 |
| GNL2 | SEC11A |
| FGF2 | DIMT1 |
| NMNAT2 | TM2D2 |
| KIF20A | FST |
| CENPI | GBE1 |
| DDX1 | PROM1 |
| XXYLT1 | CD44 |
| GPR176 | POU5F1 |
| BBS9 | ABCG2 |
| C14orf166 | SOX2 |
| BOD1 | NANOG |
| CDC123 | ALDH1A1 |
| SNRPD3 | CCL2 |
| FAM118B | MYC |
| DPH3 | KLF4 |
| EIF2B3 | |

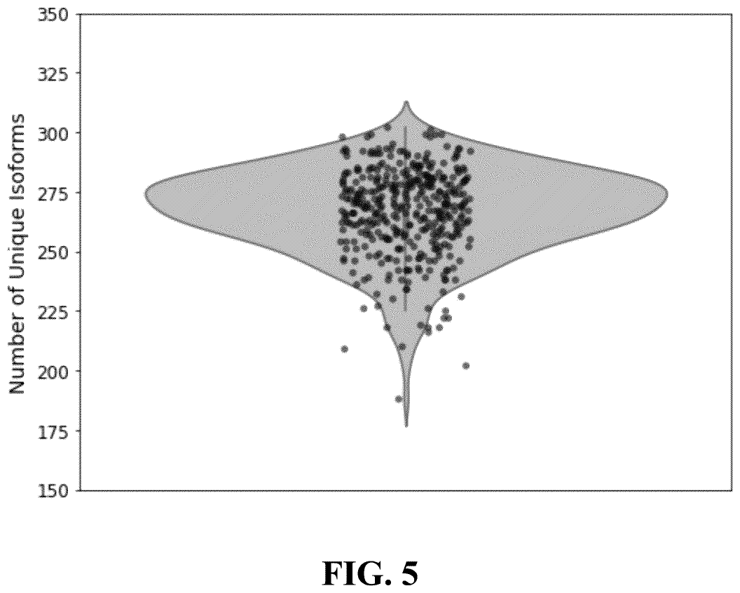

Isoforms expressed in less than 10% of samples were not included in the analysis of the present example, leaving a total of 352 features. FIG. 5 shows the distribution of unique isoforms for each patient, according to some embodiments. RSEM isoform expression data was then log 2 transformed and z-scored, according to some embodiments.

In the present example, patients with stage I STAD were defined as low-stage and patients with stage II, II, and IV STAD were defined as high-stage. In the present example, the TSVdb and TCGA report number stage, rather than TNM stage. Synthetic Minority Over-sampling Technique (SMOTE) was used to generate synthetic samples to account for classification imbalance. SMOTE creates new instances by interpolating between neighboring minority samples in the features space. The samples were randomly divided into training and testing groups at a 6:4 split. Support vector machine (SVM), ElasticNet, GradientBoosting, and Random Forest were implemented. In the present example, SVMs create a hyperplane (a line in linear kernels, planes in multi-dimensional data) to maximize the distance between data points of different classes. ElasticNet is a unique type of linear regression that can balance feature selection and coefficient shrinkage to prevent overfitting. GradientBoosting is an iterative method that builds new decision trees based on the errors of the previous tree. Random Forest is an ensemble method that builds multiple decision trees based on random subsets of the data and features, then averages their predictions. These algorithms were chosen for their ability to handle complex data and rank the importance of features.

In the present example, patients without survival data were excluded from survival analysis, leaving a total of 316 samples with 5-year survival data. The samples were randomly divided into training and testing groups at an 8:2 split. Survival SVM with RBF and linear kernels, Random Survival Forest, GradientBoosting Survival, and ElasticNet Survival algorithms were implemented. These methods are extensions of the models used for stage classification, to accommodate right-censored time-to-event data.

The algorithms of the present example were chosen for their ability to handle complex data and rank features. Hyperparameters for all stage classification models and survival models were tuned with GridSearch and metrics were evaluated with 5-fold cross validation. Final hyperparameters for all stage classification models and survival models can be found in Table 2.

TABLE 2
Final tuned hyperparameters for prediction models

| Model | Hyperparameters |
|---|---|
| Stage Classification Models | |
| SVM | C = 1.0, kernel = ‘rbf’, gamma = ‘scale’ |
| ElasticNet | alpha = 1.0, l1_ratio = 0.25, max_iter = |
| | 1000, |
| GradientBoosting | learning_rate = 0.1, max_iter = 100, |
| | max_depth = None |
| Random Forest | n_estimators = 100, max_depth = None, |
| | max_features = ‘sqrt’, |
| | min_samples_split = 2 |
| Survival Analysis Models | |
| Linear Survival SVM | alpha = 0.01, rank_ratio = 1.0, max_iter = |
| | 1000 |
| RBF Survival SVM | alpha = 0.01, rank_ratio = 1.0, gamma = |
| | None, max_iter = 100 |
| Random Survival Forest | n_estimators = 500, max_depth = 5, |
| | min_samples_split = 6 |
| GradientBoosting Survival | learning_rate = 0.1, n_estimators = 50, |
| | max_depth = 3 |
| ElasticNet Survival | l1_ratio = 0.1, alpha_min_ratio = 0.01 |

For stage classification, accuracy was used to tune models. Concordance index (C-index) was used to tune survival models, which is defined as the ratio of correctly ordered (concordant) pairs to comparable pairs. A comparable pair is concordant if the predicted risk is higher for the subject with lower survival time. Top performing trained models were then evaluated on the test data.

The ML algorithms were applied using Python 3.9 and the scikit-learn 1.0.2, scikit-survival 0.21.0, and imbalanced-learn 0.12.3 libraries. MSigDB was used for gene enrichment analysis.

### Results

### Model Performance

In one example, stage classification models were trained and evaluated. Area under the curve (AUC) values were used to assess predictive performance, along with accuracy, recall, and F1 score. Table 3 shows the performance metrics of all stage classification models that were evaluated.

TABLE 3
Performance metrics for stage classification models.

| Model | AUC | Accuracy | Recall | F1 |
|---|---|---|---|---|
| SVM | 0.899 | 0.958 | 1.00 | 0.984 |
| ElasticNet | 0.903 | 0.943 | 0.947 | 9.97 |
| GradientBoosting | 0.849 | 0.975 | 0.993 | 0.987 |
| Random Forest | 0.922 | 0.968 | 1.00 | 0.984 |

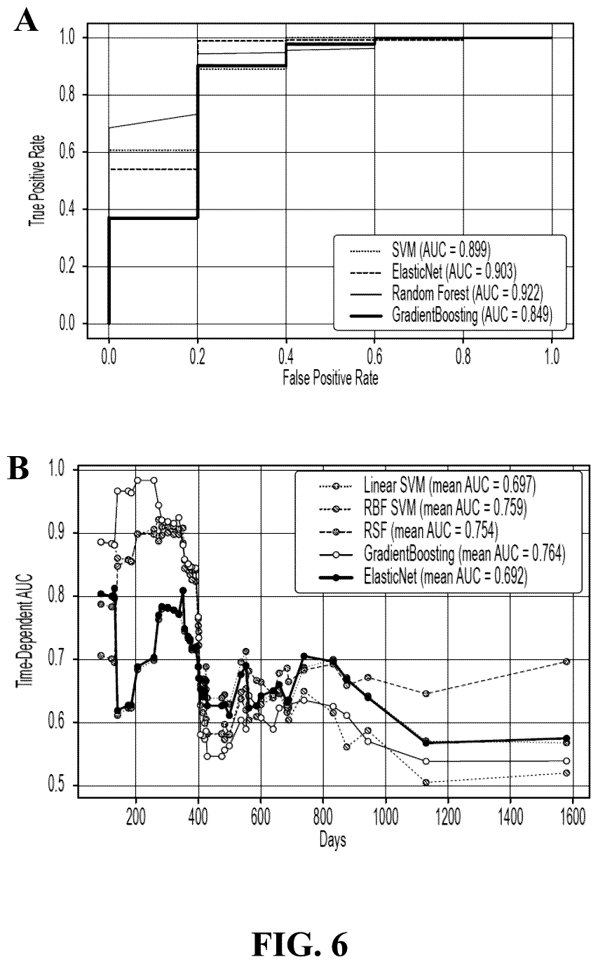

In one example, the AUC for high/low-stage predictions of the stage classification models ranged from 0.849 to 0.922 (FIG. 6A). Random Forest was the highest performing model (AUC=0.922, Accuracy=0.968, Recall=2.0, F1=0.984).

In one example, survival models were trained on 5-year survival data. Mean time-dependent AUC was used to rank model performance, as predictive performance of survival models varies at each time point. The mean 5-year AUC for survival models ranged from 0.692 to 0.764. GradientBoosting Survival was the highest performing model (Mean 5-year AUC=0.764, C-index=0.705). For all survival models, time-dependent AUC dropped significantly for predictions after ˜400 days (FIG. 6). Mean 1-year AUC was also calculated to better assess model performance for survival predictions within 1 year. Table 3 lists mean 5-year AUC, mean 1-year AUC, and C-index for all survival models evaluated. All models performed better with predictions at 1 year than at 5 years and GradientBoosting Survival showed the best performance with mean 1-year AUC of 0.945.

### Gene Enrichment Analysis

Feature importance was extracted from the GradientBoosting Survival model, the top performing survival model, and gene enrichment analysis was performed on the genes corresponding to the an example of 100 features (Table 4) to determine functional importance using Gene Ontology (GO) term enrichment and Kyoto Encyclopedia of Genes and Genomes (KEGG) pathway analyses.

TABLE 4
An Example of 100 features for GradientBoosting survival model

| Feature | Importance |
|---|---|
| isoform_uc011mqv_HDX | 0.083259 |
| isoform_uc001phm_DCUN1D5 | 0.065161 |
| isoform_uc001vht_DIAPH3 | 0.059694 |
| isoform_uc004eie_ARMCX2 | 0.052579 |
| isoform_uc004eid_ARMCX2 | 0.043196 |
| isoform_uc003bej_SAMM50 | 0.042809 |
| isoform_uc001vng_IPO5 | 0.041795 |
| isoform_uc003jhc_CDH6 | 0.039919 |
| isoform_uc003jpq_GPX8 | 0.038073 |
| isoform_uc003kmz_RIOK2 | 0.031059 |
| isoform_uc003pwe_HDAC2 | 0.0299 |
| isoform_uc003jhd_CDH6 | 0.027569 |
| isoform_uc002lbu_HAUS1 | 0.027343 |
| isoform_uc003qob_MTHFD1L | 0.026342 |
| isoform_uc002mtd_ZNF88 | 0.026214 |
| isoform_uc003puo_RPF2 | 0.026001 |
| isoform_uc002bof_KIF7 | 0.02433 |
| isoform_uc010bbk_EIF2AK4 | 0.023658 |
| isoform_uc003vuc_TRIM24 | 0.019572 |
| isoform_uc002blc_SEC11A | 0.018431 |
| isoform_uc003tdo_BBS9 | 0.015641 |
| isoform_uc001cmu_EIF2B3 | 0.015184 |
| isoform_uc002blb_SEC11A | 0.01366 |
| isoform_uc003hui_ADH5 | 0.013532 |
| isoform_uc002vfy_XRCC5 | 0.013241 |
| isoform_uc001ctu_ORC1 | 0.01257 |
| isoform_uc003guq_UBE2K | 0.01092 |
| isoform_uc003gop_PROM1 | 0.009952 |
| isoform_uc001vmv_UGGT2 | 0.008901 |
| isoform_uc011cvo_NREP | 0.008338 |
| isoform_uc003huk_ADH5 | 0.008103 |
| isoform_uc004eif_ARMCX2 | 0.007711 |
| isoform_uc001tfn_SLC25A3 | 0.007631 |
| isoform_uc001vmt_UGGT2 | 0.006085 |
| isoform_uc003udt_CCL26 | 0.005974 |
| isoform_uc001gqd_NMNAT2 | 0.005594 |
| isoform_uc010wbw_UTP6 | 0.005338 |
| isoform_uc001uws_TRPC4 | 0.004451 |
| isoform_uc003jiw_RAD1 | 0.004107 |
| isoform_uc003yph_HAS2 | 0.004032 |
| isoform_uc010fqi_HAT1 | 0.003578 |
| isoform_uc003pje_BCKDHB | 0.003365 |
| isoform_uc001tfp_SLC25A3 | 0.003324 |
| isoform_uc001hwz_TBCE | 0.003186 |
| isoform_uc003qoa_MTHFD1L | 0.003097 |
| isoform_uc003jpr_GPX8 | 0.003094 |
| isoform_uc003ysi_MYC | 0.002899 |
| isoform_uc001cmw_EIF2B3 | 0.002647 |
| isoform_uc001qte_GDF3 | 0.002524 |
| isoform_uc010yro_MTHFD2 | 0.002505 |
| isoform_uc001vhv_DIAPH3 | 0.002405 |
| isoform_uc004fay_ATP11C | 0.00227 |
| isoform_uc002skk_MTHFD2 | 0.002112 |
| isoform_uc003xmm_TM2D2 | 0.002072 |
| isoform_uc004art_IARS | 0.002056 |
| isoform_uc0111vh_TEX10 | 0.002009 |
| isoform_uc003pql_ASCC3 | 0.001876 |
| isoform_uc001cmt_EIF2B3 | 0.001803 |
| isoform_uc003pmc_RARS2 | 0.00179 |
| isoform_uc001mlf_RRAS2 | 0.001677 |
| isoform_uc001xdx_DACT1 | 0.001611 |
| isoform_uc003gou_PROM1 | 0.001441 |
| isoform_uc001cth_RAB3B | 0.001163 |
| isoform_uc010tik_IPO5 | 0.00108 |
| isoform_uc003got_PROM1 | 0.001076 |
| isoform_uc003inz_PLRG1 | 0.001014 |
| isoform_uc003gos_PROM1 | 0.001007 |
| isoform_uc002gks_PFAS | 0.000942 |
| isoform_uc004bas_TEX10 | 0.000941 |
| isoform_uc011cvp_NREP | 0.000889 |
| isoform_uc003nsv_POU5F1 | 0.000875 |
| isoform_uc001zko_EIF2AK4 | 0.000853 |
| isoform_uc003pme_RARS2 | 0.000813 |
| isoform_uc001mvw_CD44 | 0.000788 |
| isoform_uc003slw_SNX8 | 0.000744 |
| isoform_uc003ork_MED20 | 0.000697 |
| isoform_uc002rci_MYCN | 0.000628 |
| isoform_uc010kil_MTHFD1L | 0.000414 |
| isoform_uc003tdq_BBS9 | 0.000252 |
| isoform_uc003fkx_SOX2 | 0.00025 |
| isoform_uc002jxk_TBC1D16 | 0.000246 |
| isoform_uc010pxq_TBCE | 0.000238 |
| isoform_uc002uhi_HAT1 | 0.000224 |
| isoform_uc010ujd_SLC24A1 | 0.000215 |
| isoform_uc004fba_ATP11C | 0.000214 |
| isoform_uc011cqi_SKIV2L2 | 0.000192 |
| isoform_uc011aju_SNRPD3 | 0.000186 |
| isoform_uc001amk_ICMT | 0.000172 |
| isoform_uc003hbs_PAICS | 0.000163 |
| isoform_uc003hrg_ABCG2 | 0.000154 |
| isoform_uc010res_CD44 | 0.000141 |
| isoform_uc002rwd_MSH6 | 0.000137 |
| isoform_uc001xbt_DLGAP5 | 0.000136 |
| isoform_uc003tbm_GARS | 0.00012 |
| isoform_uc001zkn_EIF2AK4 | 0.000113 |
| isoform_uc001mwc_CD44 | 0.000104 |
| isoform_uc003pwd_HDAC2 | 0.000103 |
| isoform_uc001vne_IPO5 | 9.47E−05 |
| isoform_uc003jvp_CENPH | 9.16E−05 |
| isoform_uc010ssv_HMGA2 | 8.37E−05 |

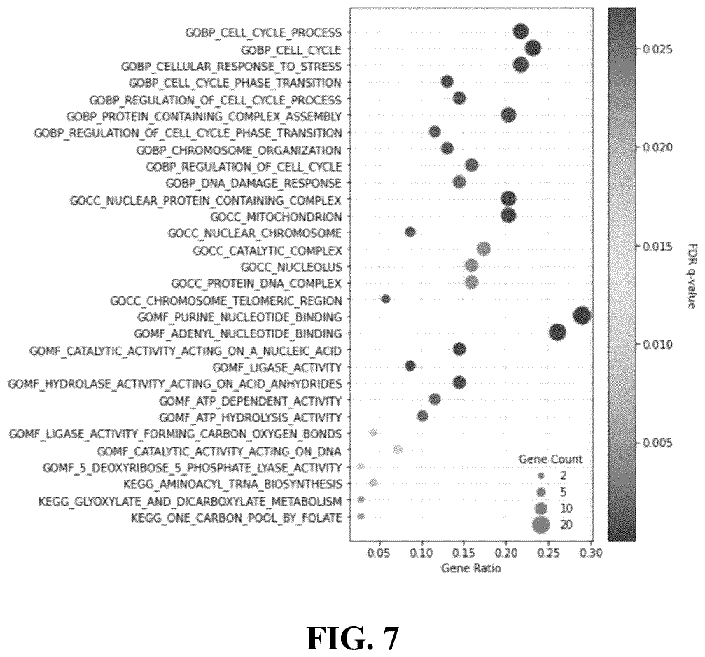

FIG. 7 shows the significant enriched gene sets with false discovery rate (FDR) less than 0.05, in one example. Gene sets involving cell cycle, cell metabolism, and nucleotide activity were all significantly enriched, in one example. GO: Molecular Function gene set “Purine Nucleotide Binding,” GO: Molecular Function gene set “Adenyl Nucleotide Binding,” and GO: Biological Process gene set “Cell Cycle Process” were the gene sets with most significant overlaps (FDR q-value=5.10e-8, 7.39e-8, and 1.67e-5, respectively), observed in one example.

### DISCUSSION

This example presents the developed and compared the performance of ML classification models and ML survival models to predict clinical stage and survival in STAD by using cancer stemness-related isoform expression data. For prediction of low/high stage STAD, the Random Forest classifier achieved the best overall performance. For survival predictions, the GradientBoosting survival model demonstrated the best overall performance, both at predicting 1-year and 5-year survival.

The findings of the present example suggest that both stage classification and survival analysis with multi-dimensional isoform expression data represent complex, highly non-linear problems. This is particularly evident with survival model performance, where the non-linear and tree-based models (RBF Survival SVM, Random Survival Forest, GradientBoosting Survival) all performed better than linear and regression-based approaches (Linear Survival SVM, ElasticNet Survival). These findings align with prior research that have shown that complex ML models are better suited for high-dimensional data and perform better than traditional methods, such as the Cox proportional hazards model.

Currently, most existing ML models for cancer survival prediction are classification models. While classification models aim to predict the probability of experiencing an event (e.g., death), they do not provide any temporal information for patient outcomes. In contrast, survival analysis models are more complex, as they aim to predict a time-to-event in addition to a binary outcome. Survival analysis is also complicated by censored survival data, where patient data may be missing outcome data due to time constraints or loss to follow-up. This may explain the significant decrease in predictive performance of all survival models after ˜400 days, as there is insufficient survival data past that time due in part to the low survival rates of STAD.

Notwithstanding these challenges, the performance of the GradientBoosting Survival model of the present example was comparable to, or higher than, that of other ML studies predicting cancer survival and gastric cancer survival, where C-index ranged from 0.57 to 0.90 and mean AUC ranged from 0.825 to 0.859. Notably, these studies generally use a combination of clinical and imaging data and employ complex deep-learning architectures. The data of the present example shows that cancer-stemness related isoform expression can be used to predict survival with comparable performance to more complex models, despite relying solely on transcriptomic data. This demonstrates the potential of cancer-stemness related isoform expression as a powerful biomarker for survival prediction in cancer.

In the present example, functional analysis revealed that the most influential features for model performance were isoforms of genes involved in nucleotide binding, cell cycle, and cellular metabolism. The GO: Molecular Function gene set “Purine Nucleotide Binding” showed the most significant overlap, with 20 overlapping genes. These include: RIOK2, which codes for a kinase involved in ribosome maturation that has been implicated in tumor cell growth and metastasis; MTHFD1L, which codes for a protein involved in the de-novo synthesis of purines that has been shown to promote proliferation in colorectal cancer and hepatocellular cancer; and KIF7, which codes for a kinesin involved in the sonic hedgehog signaling pathway whose abnormal expression is linked to poor prognosis in multiple cancers. The present example evaluates isoform expression, providing a more comprehensive and interpretable understanding of alternative splicing through its influence on isoform generation. By analyzing isoform-level expression rather than isolated splice events, the present example captures the cumulative effects of splicing mechanisms on patient prognosis, revealing isoform-specific signatures that are often masked in splicing event analysis. As such, the findings underscore how isoform-based analyses can be used for the discovery of novel prognostic markers and therapeutic targets.

The examples of the present disclosure demonstrate strong performance in predicting stage and survival in STAD based on developed predictive models. The findings of the present example show that cancer stemness-related isoform expression can serve as a valuable, standalone predictor of cancer outcomes, emphasizing its potential as a robust biomarker for prognosis and identifying potential therapeutic targets.

What is claimed is:

1. A system for guiding clinical decision-making for potential cancer patients comprising:
a first memory having stored thereon an electronic medical record database framework including medical data for a given patient;
a communication system connected to the memory to allow credentialed users at remote workstations to input new medical data into and retrieve existing medical data from the first memory;
a processor communicatively connected to the first memory and a second memory; and
wherein the second memory has software instructions stored thereon which, when executed by the processor, cause the system to:
determine an indication of a preliminary cancer diagnosis of a given type of cancer for the given patient from the medical data stored on the first memory;
confirm an order for a biopsy and associated transcriptional sequencing analysis of the biopsy;
receive results of the transcriptional sequencing analysis, the results comprising transcriptomic data representative of isoform and gene expression of cells of the biopsy;
extract a set of isoform expression information from the transcriptomic data, based on one or more gene cancer stemness-related signatures specific to the preliminary cancer diagnosis, to create a cancer stemness isoform dataset for the patient;
process the cancer stemness isoform dataset for the patient using a first machine learning model to obtain a baseline current cancer stage prediction for the patient, the first machine learning model having been trained on cancer patient data from patients confirmed to have the given type of cancer and configured to output a prediction of cancer stage classification;
process the cancer stemness isoform dataset for the patient using a second machine learning model to obtain baseline survival prediction information for the patient, the first machine learning model having been trained on cancer patient data from patients confirmed to have the given type of cancer and configured to output survival prediction information over a period of time after diagnosis;
store the current cancer stage prediction and the survival prediction information in the first memory with the medical data for the given patient;
output via the communication system an indication to a healthcare provider of the cancer stage prediction, the survival prediction information and at least one a treatment recommendation based thereon.

2. The system of claim 1 wherein the first machine learning model generates the current cancer stage prediction based solely on an input dataset consisting essentially of the cancer stemness isoform dataset.

3. The system of claim 1 wherein the second machine learning model generates the survival prediction information based solely on an input dataset consisting essentially of the cancer stemness isoform dataset.

4. The system of claim 1 wherein the one or more gene cancer stemness-related signatures comprise a cancer stemness signature of a set of genes and a set of cancer stemness markers.

5. The system of claim 4 wherein the given type of cancer is gastric adenocarcinoma, and the one or more gene cancer stemness-related signatures were generated by removing from a 109 gene cancer stemness signature and a set of 10 cancer stemness markers information for isoforms present in less than a threshold percentage of biopsies of other known patients having the given type of cancer.

6. The system of claim 1 wherein the software further causes the system to:
determine an indication that the given patient underwent a treatment for the given type of cancer;
confirm a second biopsy and second transcriptional analysis for the given patient;
generate a second current cancer stage prediction for the patient using the first trained machine learning model and a second survival prediction information for the patient using the second trained machine learning model;
compare the baseline current cancer stage prediction to the second current cancer stage prediction;
compare the baseline survival prediction information to the second survival prediction information; and
based on the comparisons, output a notification of change to the healthcare provider and an assessment of impact of the treatment.
